(19) United States
(12) Patent Application Publication
(10) Pub. No.: US 2018/0303347 A1
(43) Pub. Date: Oct. 25, 2018

(54) Raman Imaging Systems and Methods

(71) Applicant: The Regents of the University of California, Oakland, CA (US)

(72) Inventors: Warren S. Grundfest, Los Angeles, CA (US); Oscar M. Stafsudd, Los Angeles, CA (US); Asael Papour, Los Angeles, CA (US); Zachary Taylor, Los Angeles, CA (US)

(73) Assignee: The Regents of the University of California, Oakland, CA (US)

(21) Appl. No.: 15/530,266

(22) PCT Filed: Jun. 18, 2015
(86) PCT No.: PCT/US2015/036518

§ 371 Date: Dec. 16, 2016

Related U.S. Application Data
(60) Provisional application No. 62/014,003, filed on Jun. 18, 2014.

(51) Int. Cl. A61B 5/00; G01J 3/44; H04N 5/33; G01J 3/28

(52) U.S. Cl. CPC A61B 5/0075; G01J 3/44; A61B 5/4504; G01J 3/2823; H04N 5/332

(57) ABSTRACT

Systems and methods for biocompatible tissue characterization using Raman imaging are provided. The systems and methods utilize Raman systems tuned to monitor spectral wavelengths characteristic of target types of tissue to monitor constituents of that tissue in biological systems and samples. The Raman systems may be tuned to monitor the Raman signature for the formation of the chemical bonds that join phosphorous and oxygen (PO) atoms, such that the formation of hydroxyapatite may be monitored and used to determine the presence of bone formation in a sample, such as, for example, biological tissue.

### STATEMENT OF FEDERAL FUNDING

This invention was made with Government support under W81XWH-12-2-0075, awarded by the U.S. Army, Medical Research and Materiel Command. The Government has certain rights in the invention.

### TECHNICAL FIELD

The present disclosure is directed to Raman imaging methods and systems; and more particularly to Raman imaging methods and systems for fast biocompatible tissue characterization.

### BACKGROUND OF THE DISCLOSURE

Biocompatible imaging of tissue, particularly imaging of tissue in vivo is difficult, because most standard techniques are time-consuming, require expensive machinery, and/or is destructive in nature. As a result, there are many disorders that cannot be feasibly detected or imaged via traditional methods. For example, bone growth in flesh is an undesirable outcome that can occur in open wounds where trauma to a limb is severe. Such bone growth, if not treated, can cause the wound to fail and ultimately lead to amputation. While early detection is crucial in failed wounds, current technologies, including X-ray and MRI, are limited and do not offer the resolution and sensitivity that are required to provide early detection in such cases. Accordingly, a need exists for improved imaging techniques capable of providing fast, inexpensive, biocompatible detection, such as, detection of bone growth in failed wounds at a stage early enough to allow for appropriate treatment.

### BRIEF SUMMARY

The present disclosure provides embodiments directed to systems and methods for biocompatible tissue characterization using Raman imaging.

In some embodiments the disclosure is directed to biocompatible imaging Raman system including:
a sample containing therein at least one substance emitting at least one Raman signal over at least one unique wavelength when the substance is excited,
an illumination source in radiative alignment with the sample, the illumination source illuminating the sample over at least one excitation wavelength to excite the sample thereby stimulating the emission of the at least one Raman signal from the substance,
an imager in optical alignment with the sample, the imager being tuned to a detection wavelength capturing a signal containing at least the at least one Raman signal from the substance along with a background emission,
one or both of the excitation wavelength of the illumination source and the detection wavelength of the imager being tunable over at least two wavelengths, wherein at least one of the at least two wavelengths includes the at least one Raman signal and at least one of the at least two wavelengths omits the at least one Raman signal such that only the background emissions are captured by the imager, and
a signal processor for subtracting the signal containing the at least one Raman signal from the signal omitting the at least one Raman signal to obtain a data set containing only the at least one Raman signal.

In other embodiments, the illumination source is one of either coherent or non-coherent and is selected from the group consisting of a laser diode and a light emitting diode, and wherein the imager is selected from the group consisting of PMT, CCD, iCCD, EMCCD and CMOS imagers.

In still other embodiments, the substance is hydroxyapatite and the at least one Raman signal arises from the excitation of the phosphorous-oxygen bonds within the hydroxyapatite.

In yet other embodiments, the excitation wavelength of the illumination source is tunable over at least two wavelengths.

In still yet other embodiments, the detection wavelength of the imager is tunable over at least two wavelengths. In some such embodiments the imager incorporates one or more filters for tuning the detection wavelength. In other such embodiments the filters are one of either illumination rejection or narrow pass-band filters.

In still yet other embodiments, the sample contains at least two distinct Raman signals.

In still yet other embodiments, the illumination source includes an array of radiative emitters arranged to simultaneously illuminate a target area, and wherein the imager has a field of view sufficiently large to capture the entire target area in a single capturing step.

In other embodiments the disclosure is directed to a method of performing biocompatible imaging Raman including:
providing a sample containing therein at least one substance emitting at least one Raman signal over at least one unique wavelength when the substance is excited,
illuminating the sample over at least one excitation wavelength to excite the sample to stimulate the emission of the at least one Raman signal from the substance,
imaging the sample at a detection wavelength capturing a signal containing at least the at least one Raman signal from the substance along with a background emission,
tuning one of either the excitation wavelength of the illumination source or the detection wavelength of the imager over at least a second wavelength that omits the at least one Raman signal such that the at least one Raman signal from the substance is not captured,
reimaging the sample at the second wavelength to obtain a signal lacking the Raman signal, and
subtracting the signal containing the at least one Raman signal from the signal lacking the at least one Raman signal to obtain a data set containing only the at least one Raman signal.

In some embodiments, the tuning includes altering the excitation wavelength such that the at least one Raman signal from the substance radiates at a wavelength different from the detection wavelength.

In other embodiments, the tuning includes altering the detection wavelength such that the unique wavelength of the Raman signal and the detection wavelength differ. In some such embodiments altering the detection wavelength includes using one or more wavelength filters. In other such embodiments the filters are one of either illumination rejection or narrow pass-band filters.

In still other embodiments, the sample contains at least two Raman signals over at least two distinct wavelengths, and wherein the method further includes imaging, tuning and reimaging to capture each of the at least two distinct Raman signals separately. In some such embodiments, the at least two Raman signals arise from at least two distinct substances.

In yet other embodiments, the illumination source is provided by one of either coherent or non-coherent and is selected from the group consisting of a laser diode and a light emitting diode, and wherein the imaging is provided by an imager selected from the group consisting of a PMT, CCD, iCCD, EMCCD and CMOS imagers.

In still yet other embodiments, the substance is hydroxyapatite and the at least one Raman signal arises from the excitation of the phosphorous-oxygen bonds within the hydroxyapatite.

In still yet other embodiments, the illuminating includes simultaneously illuminating a target area, and wherein the imaging comprises capturing the entire target area in a single capturing step.

Additional embodiments and features are set forth in part in the description that follows, and in part will become apparent to those skilled in the art upon examination of the specification or may be learned by the practice of the invention. A further understanding of the nature and advantages of the present invention may be realized by reference to the remaining portions of the specification and the drawings, which forms a part of this disclosure.

### BRIEF DESCRIPTION OF THE DRAWINGS

The description will be more fully understood with reference to the following figures and data graphs, which are presented as various embodiments of the disclosure and should not be construed as a complete recitation of the scope of the disclosure, wherein:

FIG. 1 provides a schematic of imaging Raman systems in accordance with exemplary embodiments of the invention.

FIG. 2 provides a flowchart of methods of performing imaging Raman system in accordance with exemplary embodiments of the invention.

FIG. 3a provides a flowchart of methods of performing illumination tuned imaging Raman system in accordance with exemplary embodiments of the invention.

FIG. 3b provides a flowchart of methods of performing detection tuned imaging Raman system in accordance with exemplary embodiments of the invention.

FIG. 4 provides a data graph of spectra taken using imaging Raman systems and methods in accordance with exemplary embodiments of the invention.

### DETAILED DESCRIPTION

The present disclosure may be understood by reference to the following detailed description, taken in conjunction with the drawings as described below. It is noted that, for purposes of illustrative clarity, certain elements in various drawings may not be drawn to scale.

In accordance with the provided disclosure and drawings, systems and methods for biocompatible tissue characterization using Raman imaging are provided. Many embodiments of the imaging Raman systems and methods are adapted to acquire parallel full field images of tissue in real time without resolving or scanning Raman spectra, to capture the location of specific tissue structures. In many embodiments the systems and methods are tuned to spectral wavelengths characteristic of target types of tissue to monitor constituents of that tissue in biological systems and samples. In some embodiments the systems and methods are tuned to monitor the Raman signature for the formation of the chemical bonds that join phosphorous and oxygen (PO) atoms. In some such embodiments, the Raman systems and methods are thus utilized to monitor the formation of the hydroxyapatite (HO) complex. In still other such embodiments hydroxyapatite formation is observed to monitor/image bone formation and growth in a biocompatible manner.

Raman spectroscopy is a powerful technique capable of detecting and imaging select substances by generating information on the bonds and the structure within a material. Traditional Raman detection involves point measurement and acquisition of Raman spectra using a spectrometer. The spectra obtained from such an acquisition procedure is then compared to a known spectra database to find a match. These spectra can serve as a chemical fingerprint to identify constituents of a sample, such as for example tissue in biological samples. In traditional Raman imaging detection is performed by raster scanning the sample area point by point to create a color map for the different constituents.

These traditional methods are extremely accurate however; they are time-consuming processes that cannot be applied in vivo due to several limiting factors. Two prominent problems are artifacts caused by the natural movements of the patients, and the high fluence levels of the illumination source that can cause dehydration of the tissue, denaturation of proteins, and destruction of other constituents. These patient sampling and biocompatibility issues mean that traditional Raman techniques, though a potentially valuable tool, are currently not practical for in vivo applications. Accordingly, in many embodiments imaging Raman systems and methods are adapted to acquire parallel full field images of tissue in real time without resolving or scanning Raman spectra, to capture the location of specific tissue structures, such as, for example, bone, using the unique spectral signature characteristic of the target tissue type, such as, for example, the PO bond associated with growth of the HO complex.

### Imaging Raman System

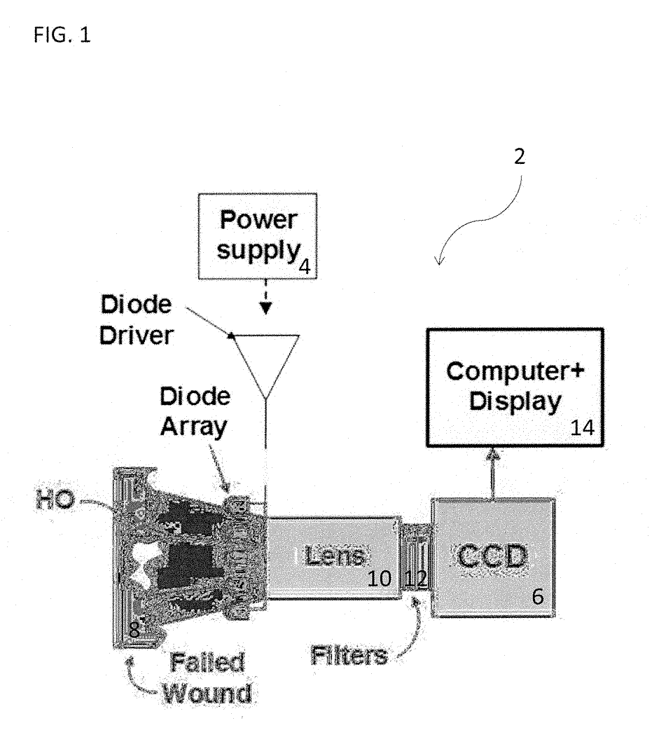

In many embodiments imaging Raman systems are provided that are adapted to acquire parallel full field images of tissue in real time without resolving or scanning Raman spectra. FIG. 1 provides a schematic according to some embodiments of such systems. As shown, the Raman system (2) generally comprises an illumination source (4) that may be tuned to emit over one or more spectral wavelengths of interest, and that in many embodiments is capable of full field parallel illumination of a sample without rastering, such as for example by utilizing one or an array of more than one light source, which may be coherent or incoherent, such as, for example, a laser diode or other light emitting diodes (LED). In many embodiments the light source is capable of emitting in the near infra-red (IR). Such system embodiments also include an imager (6), such as for example a photomultiplier tube (PMT), charge coupled device (CCD), intensified charge coupled device (iCCD), electron multiplying charge coupled device (EMCCD), or complementary metal-oxide semiconductor (CMOS) imager, adapted to take a direct measurement from the entire sample (8) without rastering or resolving a Raman spectra, and suitable imaging optics (10) and spectral filters (12), such as for example pass-band and optical rejection (notch) filters to condition the signal prior to imaging such that only the desired spectral wavelength is imaged by the imager, and sources of noise, such as signal from the illumination source and non-Raman sources, may be rejected.

In one exemplary embodiment, the Raman system is adapted to detect and image the growth of bone, by monitoring spectral frequencies associated with the formation of the PO bonds associated with the creation of HO. In one such embodiment, the illumination source (4) is a near infrared LED or laser adapted to emit at wavelengths between 700 and 800 nm (and in some embodiments at 785 nm and 781.5 nm), and a CCD spectral imager in optical communication with the sample (8). The imaging optic (10) and spectral filters (12) are positioned in optical alignment between the illuminated sample (8) and the CCD imager. In particular, the spectral filters may at least include an optical rejection (or notch) filter and a narrow pass-band filter tuned to the spectral wavelength of the target substance's emission (in these exemplary embodiments the Raman signature of PO may be measured in shifted wavenumbers (cm⁻¹) and is located 960 cm⁻¹ from the illumination source, accordingly for a light source operating at 785 nm and 781.5 nm the Raman shift for PO would occur at 849 nm and 845 nm, respectively) adapted to reject the signals from the illumination source and from any non-Raman sources.

Although specific imaging Raman system embodiments adapted for use in detecting bone and bone growth in a tissue sample are described above, it should be understood that the system may be adapted for use in detecting and imaging any sample (e.g., biological material) capable of generating a unique Raman signal. For the purposes of this disclosure the term sample means both materials (biological or non-biological) removed from a body and imaging sample regions deposed in-situ on or within a target body (e.g., a human patient). Such adaption requiring only the adoption of appropriate control and imaging spectral wavelengths, and the use of suitable illumination sources and filters, such that the unique Raman signal can be acquired and isolated by the imager in accordance with the methodologies discussed below.

Likewise, although the above embodiments describe a system for isolating at a single wavelength, it should be understood that the system could be adapted to image more than one spectral wavelength of interest. In such embodiments, the system would include additional filters adapted to image these additional spectral wavelengths.

In addition, though not discussed above, embodiments of the systems may also include signal processors (14) adapted to process the images obtained from the imager to obtain an image of the sample showing materials that have an emission at the specific wavelength of interest. For example, in many embodiments such systems may include a processor capable of subtracting two unique images of a sample to obtain signals from a Raman signal at one or more desired wavelengths.

### Imaging Raman Methodology

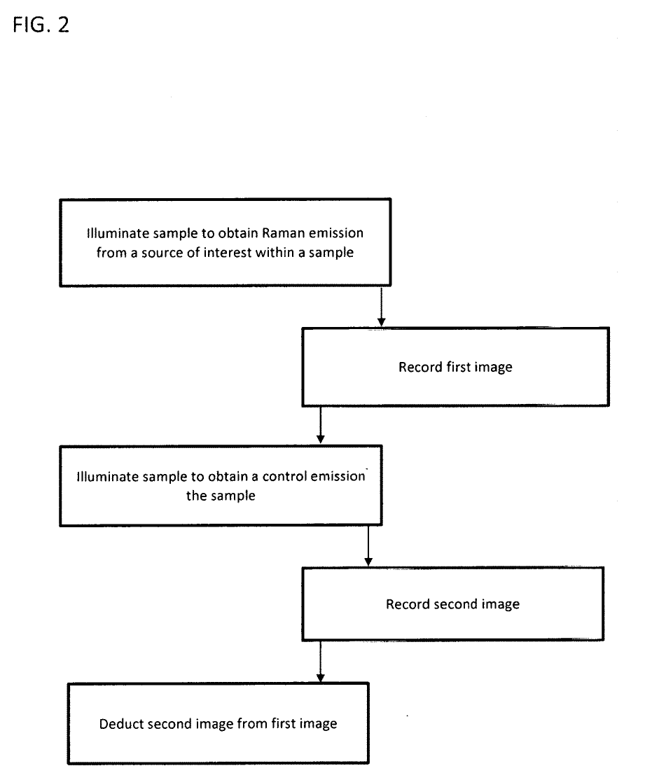

In many embodiments, imaging Raman methods are provided that are adapted to acquire parallel full field images of tissue in real time without resolving or scanning Raman spectra. FIG. 2 provides a flowchart according to some embodiments of such methods. As shown, in many embodiments a sample of interest is illuminated to produce a Raman emission from a source of interest within the sample. The emission from the sample is filtered to reject signal from the illumination source and any non-Raman source, and an image is taken of the sample emission by an imager, such as a CCD, iCCD, EMCCD, or CMOS. This process is then repeated at least a second time to obtain a second unique image of the resultant sample emission where the Raman emission from the source of interest within the sample is not imaged. The at least two images are then processed such as by subtracting one from the other to yield an image of the isolated Raman signal from the source of interest. It should be understood that in such embodiments the acquisition of the images are sustained until sufficient signal is obtained to render an image of the source signal.

For example, in some embodiments the source of interest is bone within a tissue sample. In such an embodiment the sample would be illuminated at least twice, once to yield a Raman signal that includes the unique Raman signal associated with PO bond formation during the creation of HO, and once under conditions that do not yield the unique Raman signal from the PO bonds. These images would then be subtracted to yield the unique Raman signal indicative of the presence HO, thus creating an intensity map of bone location within the tissue sample.

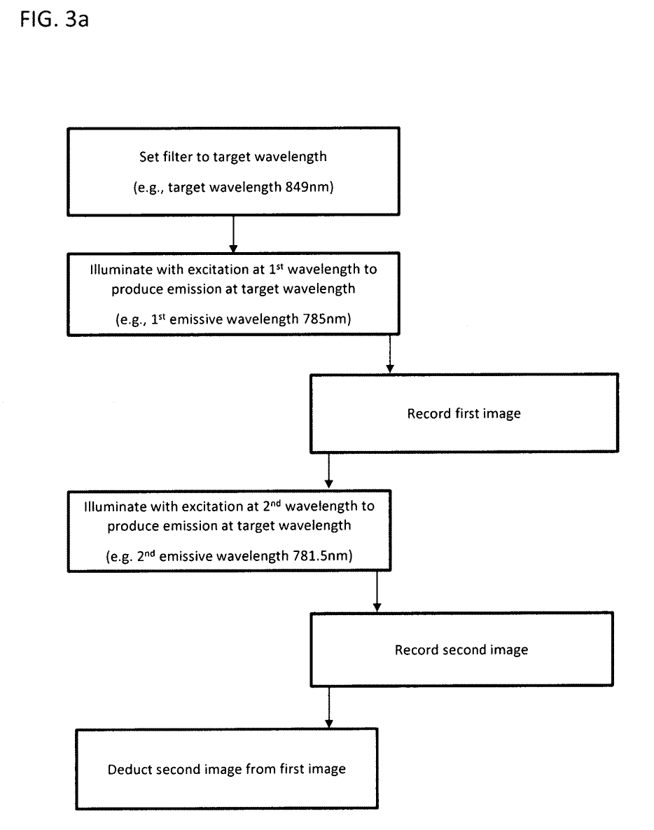

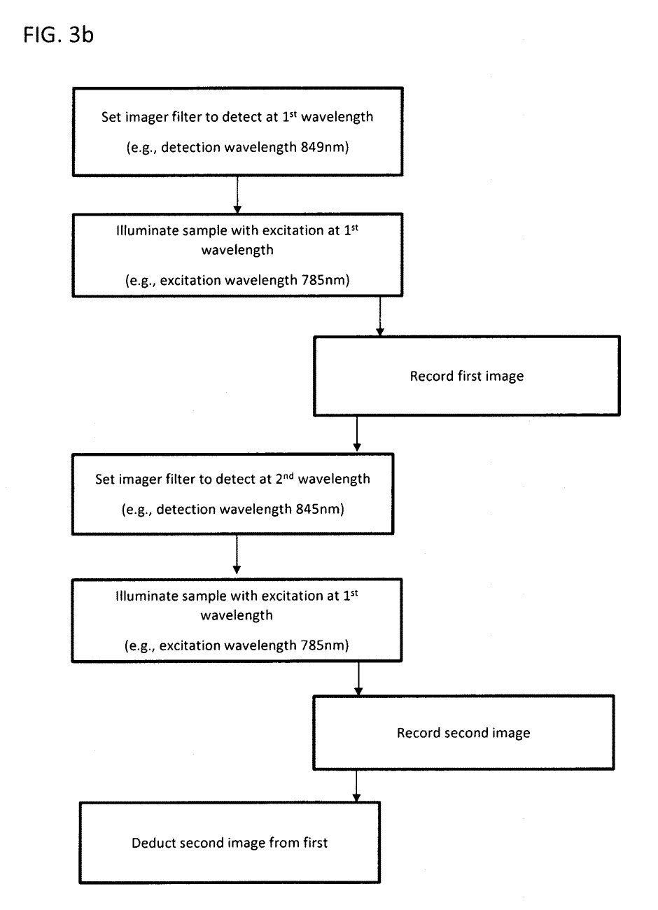

Although embodiments of generalized methods for biocompatible imaging Raman are presented above, it should be understood that, in accordance with embodiments, there are multiple methods for obtaining the at least two unique images of the sample, including, for example, illumination source wavelength tuning and detection wavelength tuning. A flowchart in accordance with embodiments incorporating an illumination source wavelength tuning method is provided in FIG. 3a. As shown, in such embodiments, the method generally comprises taking a first image of a sample being illuminated at an illumination wavelength that excites at least a Raman signal uniquely characteristic of a source of interest within the sample. For example, in embodiments directed to the detection of bone within a tissue sample, the first image may be taken with an LED illumination at a wavelength of 785 nm, which would excite Raman emissions at 849 nm (and other fluorescence signals) that are characteristic of the presence of PO bonds in HO molecules indicative of the presence of bone structures within the tissue sample. The various filters and the imager would be tuned to image signals at this characteristic wavelength (849 nm), thus recording the signal from the PO bonds.

In such embodiments, the second image is then acquired by tuning the illumination source to a wavelength that shifts the Raman signal uniquely characteristic of the source of interest to a wavelength that would be rejected by the filters of the system, meaning that the imager would only receive signals from the excitation of the sample characteristic of background fluorescence. Again, in an exemplary embodiment directed to obtaining information/imagery concerning the presence of bone in a tissue sample, the illumination source might be tuned to 781.5 nm, thus shifting the PO Raman signal to 845 nm. The imager and filters, being tuned to detect signals at 849 nm, would yield an image showing only fluorescence signals at the 849 nm window and would reject the new Raman signature of the PO bond. Once these two unique images of the sample are obtained, subtraction of the second image from the first will result in a new image that would hold only the information of the unique Raman PO signals, thus allowing for the creation of a map of the bone structure locations in the acquired field of view.

A flowchart in accordance with embodiments incorporating a detection wavelength tuning method is provided in FIG. 3b. As shown, in such embodiments, the method generally comprises taking a first image at a first wavelength that excites a Raman spectra unique to a substance of interest within the sample at a wavelength at which the imager and the imaging filters are tuned. For example, in embodiments directed to imaging bone within a tissue sample, the first image would be taken with an LED illumination at 785 nm, capturing Raman signals at 849 nm (as described in reference to the method of FIG. 3a). However, in embodiments incorporating detection wavelength tuning, the second image is acquired with the illumination source emitting at the same wavelength, but with the Imager's imaging filers being tuned to a second wavelength such that the unique Raman spectra from the source of interest is not imaged. Again, in embodiments directed to detection of bone in tissue, the imaging camera's filter might be tuned to 845 nm, while the illumination source is kept at 785 nm. Tuning the detector in this manner ensures that the unique Raman PO signal is not captured in the second image. Again, subtraction of the second image from the first results in a new image that would hold only the information of the unique Raman PO signals, and would thus provide a map of source of interest, identified by its unique Raman signal, such as, for example, the bone structures within tissue.

Although specific imaging Raman method embodiments adapted for use in detecting bone and bone growth in a tissue sample are described above, it should be understood that the system may be adapted for use in detecting and imaging any biological material having a unique Raman signal. Such adaption requiring only the adoption of appropriate control and imaging spectral wavelengths, and the use of suitable illumination sources and filters, such that the unique Raman signal can be acquired and isolated by the imager in accordance with the methodologies discussed below.

Likewise, although the above embodiments describe methods for isolating at a single Raman wavelength, it should be understood that the system could be adapted to image more than one spectral wavelength of interest. In such embodiments, the Raman methods would include additional steps adapted to image these additional spectral wavelengths.

### EXEMPLARY EMBODIMENTS

The present invention will now be illustrated by way of the following systems and methods, which are exemplary in nature and are not to be considered to limit the scope of the invention.

### Imaging Raman For Bone Detection

Many embodiments are directed to optical imaging systems that are capable of safely and reliably obtaining Raman signals from biologic tissues. In such embodiments, the systems are capable of acquiring parallel full field images of tissue without resolving or scanning Raman spectra in real-time.

An example of this capability is for Heteroptopic Ossification (HO) within a tissue sample. Bone formation involves creation of a complex structure called Hydroxyapatite, a major building block of bone. This structure has many chemical bonds that join Phosphorous and Oxygen atoms together. These bonds can be identified by their unique optical Raman signature. Accordingly, in many embodiments, the technique relies on the detection of the Phosphorous-Oxygen (PO) chemical bond, found in bone. Optical Raman signature of the Phosphorous-Oxygen (PO) chemical bond, a prevalent bond in the bone matrix, is unique and not found in significant quantities in other constituents of flesh. (The Raman signature of PO is measured in shifted wavenumbers (cm⁻¹), and is located 960 cm⁻¹ from the illumination source.) Since the emitted PO Raman signal depends on the illumination wavelength, conversion to wavelength is useful once the illumination source is known. For example, using 785 nm and 781.5 nm illumination sources, the expected Raman shifts would occur at 849 nm and 845 nm respectively.

Regardless of the specific illumination wavelength used, the unique signature from the PO bonds can serve as an indication for bone existence, which is an important capability, because bone growth in flesh is an undesirable outcome and it can occur in open wounds where trauma to the limb is severe, causing the wound to fail and ultimately leading to amputation. Thus, embodiments of the Raman systems and methods provide a capability to detect HO, its early stages of formation and early stages of bone formation outside the skeleton in flesh using this unique Raman signature, thus capturing the location of HO structures embedded in flesh. Optical imaging using Raman signatures of HO offers high resolution and high sensitivity, with ˜1 cm penetration depth, that can detect the early stages of HO formation, thus allowing for the initiation of treatment earlier in patients, leading to better patient outcomes

In exemplary embodiments, a suitable imaging Raman system would incorporate a light source, such as a laser diode or light emitting diode (LED) (preferably one capable emitting at wavelengths between 700 nm and 800 nm). Such an illumination source preferably enables full field parallel illumination of the sample with good beam uniformity and low noise. An imager, such as a CCD, iCCD, EMCCD, or CMOS imager along with appropriate filters (such as illumination rejection and narrow pass-band filters) capable of capturing two direct measurement (images) of the sample in different wavelengths allows for the mapping of the locations of HO and HO formation without resolving Raman spectra or raster scanning. The notch filter and the narrow pass-band filters are place in the optical path of the imaging optics (e.g., in front or in back) to reject the illumination source and all non-desired signals, including fluorescence. During operation, two images of the sample are acquired, each of which is recorded at different wavelengths. A subtraction of the two images reveals only the unique (PO) Raman signals, creating intensity map of bone and/or HO locations.

As discussed above, two different tuning methods may be employed to capture the two images. In illumination wavelength tuning, the first image is taken with an LED illumination at 785 nm, capturing Raman at 849 nm signals from bone structures and other fluorescence signals. (The first image being acquired until sufficient signal is reached.) The second image is then acquired with the source tuned to 781.5 nm, thus shifting the PO Raman signal to 845 nm. This image will show only fluorescence signals at the 849 nm window and reject the new Raman signature of the PO bond. Subtraction of the second image from the first will result in a new image; this image containing the isolated Raman PO signals, allowing for the mapping of the HO and/or bone structure locations in the acquired field of view. In contrast, in detection wavelength tuning, the first image is taken with an LED illumination at 785 nm, capturing Raman signals at 849 nm (same as scheme I). The second image is acquired with the camera's filter tuned to 845 nm, while the source is kept at 785 nm. This ensures that the unique Raman PO signal is not captured in the second image. Again, subtraction of the second image from the first results in a location map of bone structures, using unique Raman signatures to distinguish different tissue constituents, e.g., in HO, collecting bone PO signal to differentiate from other tissues.

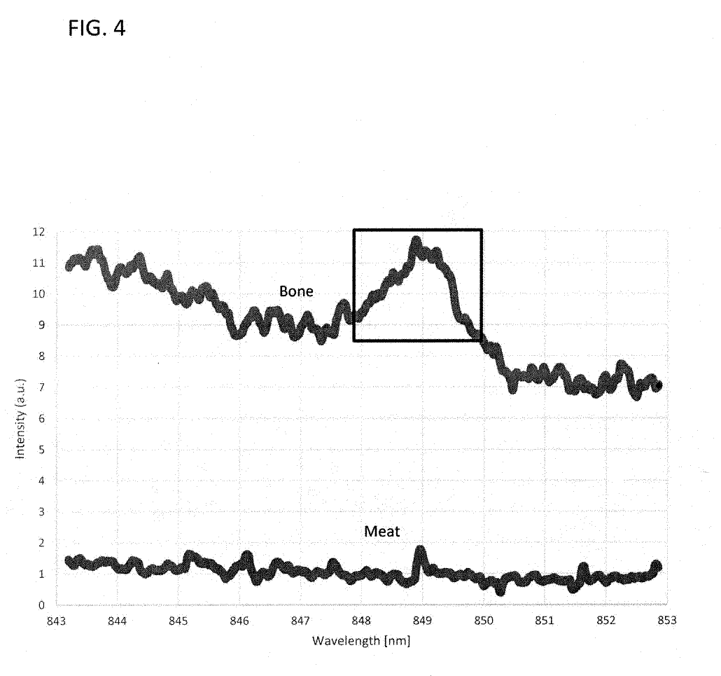

To test the viability and biocompatibility of embodiments of the imaging Raman systems and methods, test measurements were made using the system and method described above. The results of this test are summarized in data graph provided in FIG. 4. The graph shows two major points:
The bone signal is much stronger than the signal from the surrounding ‘meat’ tissue; and
The Unique Raman peak (box at 849 nm) is significant and can be used as a marker to detect the presence of bone within a surrounding tissue sample.
Using a correction/subtraction technique, the Raman signal can be isolated with suppression of the background to make a single unique readout for HO measurements.
   Using the unique signal generated in accordance with embodiments of the system and methods, it can be seen that a unique image of the signal could, likewise, be acquired using a camera system to image the locations of bone in the tissue.

These results indicate that embodiments of the systems and methods have the potential to complement and enhance current tissue imaging systems, such as, for example, X-ray (including CT), and Magnetic Resonance Imaging (MRI) that are currently used to map tissue constituents, with real time optical imaging that can characterize tissue non-expensively. In addition, in case of HO, embodiments of the systems and methods may provide early detection and mediation of:
Failed wounds (combat related trauma), that suffer from HO and results in amputation;
Surgical hip replacement complications caused by HO;
Brain or spinal cord injuries that lead to HO; and
Severe burn wound complications related to the production of HO.

Moreover, these results were obtained using a low power source, without damaging (burning or dehydrating) the muscle and bone. This has importance in the biomedical field since conventional systems tend to damage the tissue and thus have limited capacity for translation to a clinical setting. Accordingly, this technique opens the opportunity for Raman imaging use in medicine and could potentially improve patients' outcome by providing better diagnostic tools for doctors.

Having described several embodiments, it will be recognized by those skilled in the art that various modifications, alternative constructions, and equivalents may be used without departing from the spirit of the invention. Additionally, a number of well-known processes and elements have not been described in order to avoid unnecessarily obscuring the present invention. Accordingly, the above description should not be taken as limiting the scope of the invention.

Those skilled in the art will appreciate that the presently disclosed embodiments teach by way of example and not by limitation. Therefore, the matter contained in the above description or shown in the accompanying drawings should be interpreted as illustrative and not in a limiting sense. The following claims are intended to cover all generic and specific features described herein, as well as all statements of the scope of the present method and system, which, as a matter of language, might be said to fall therebetween.

What is claimed is:

1. A biocompatible imaging Raman system comprising:
a sample containing therein at least one substance emitting at least one Raman signal over at least one unique wavelength when the substance is excited;
an illumination source in radiative alignment with the sample, the illumination source illuminating the sample over at least one excitation wavelength to excite the sample thereby stimulating the emission of the at least one Raman signal from the substance;
an imager in optical alignment with the sample, the imager being tuned to a detection wavelength capturing a signal containing at least the at least one Raman signal from the substance along with a background emission;
one or both of the excitation wavelength of the illumination source and the detection wavelength of the imager being tunable over at least two wavelengths, wherein at least one of the at least two wavelengths includes the at least one Raman signal and at least one of the at least two wavelengths omits the at least one Raman signal such that only the background emissions are captured by the imager; and
a signal processor for subtracting the signal containing the at least one Raman signal from the signal omitting the at least one Raman signal to obtain a data set containing only the at least one Raman signal.

2. The imaging Raman system of claim 1, wherein the illumination source is one of either coherent or non-coherent and is selected from the group consisting of a laser diode and a light emitting diode, and wherein the imager is selected from the group consisting of PMT, CCD, iCCD, EMCCD and CMOS imagers.

3. The imaging Raman system of claim 1, wherein the substance is hydroxyapatite and the at least one Raman signal arises from the excitation of the phosphorous-oxygen bonds within the hydroxyapatite.

4. The imaging Raman system of claim 1, wherein the excitation wavelength of the illumination source is tunable over at least two wavelengths.

5. The imaging Raman system of claim 1, wherein the detection wavelength of the imager is tunable over at least two wavelengths.

6. The imaging Raman system of claim 5, wherein the imager incorporates one or more filters for tuning the detection wavelength.

7. The imaging Raman system of claim 6, wherein the filters are one of either illumination rejection or narrow pass-band filters.

8. The imaging Raman system of claim 1, wherein the sample contains at least two distinct Raman signals.

9. The imaging Raman system of claim 1, wherein the illumination source comprises an array of radiative emitters arranged to simultaneously illuminate a target area, and wherein the imager has a field of view sufficiently large to capture the entire target area in a single capturing step.

10. A method of performing biocompatible imaging Raman comprising:
providing a sample containing therein at least one substance emitting at least one Raman signal over at least one unique wavelength when the substance is excited;
illuminating the sample over at least one excitation wavelength to excite the sample to stimulate the emission of the at least one Raman signal from the substance;
imaging the sample at a detection wavelength capturing a signal containing at least the at least one Raman signal from the substance along with a background emission;
tuning one of either the excitation wavelength of the illumination source or the detection wavelength of the imager over at least a second wavelength that omits the at least one Raman signal such that the at least one Raman signal from the substance is not captured;
reimaging the sample at the second wavelength to obtain a signal lacking the Raman signal; and
subtracting the signal containing the at least one Raman signal from the signal lacking the at least one Raman signal to obtain a data set containing only the at least one Raman signal.

11. The method of claim 10, wherein the tuning comprises altering the excitation wavelength such that the at least one Raman signal from the substance radiates at a wavelength different from the detection wavelength.

12. The method of claim 10, wherein the tuning comprises altering the detection wavelength such that the unique wavelength of the Raman signal and the detection wavelength differ.

13. The method of claim 12, wherein altering the detection wavelength includes using one or more wavelength filters.

14. The method of claim 13, wherein the filters are one of either illumination rejection or narrow pass-band filters.

15. The method of claim 10, wherein the sample contains at least two Raman signals over at least two distinct wavelengths, and wherein the method further comprises imaging, tuning and reimaging to capture each of the at least two distinct Raman signals separately.

16. The method of claim 15, wherein the at least two Raman signals arise from at least two distinct substances.

17. The method of claim 10, wherein the illumination source is provided by one of either coherent or non-coherent and is selected from the group consisting of a laser diode and a light emitting diode, and wherein the imaging is provided by an imager selected from the group consisting of a PMT, CCD, iCCD, EMCCD and CMOS imagers.

18. The method of claim 10, wherein the substance is hydroxyapatite and the at least one Raman signal arises from the excitation of the phosphorous-oxygen bonds within the hydroxyapatite.

19. The method of claim 10, wherein the illuminating comprises simultaneously illuminating a target area; and
wherein the imaging comprises capturing the entire target area in a single capturing step.
